(19) United States
(12) Patent Application Publication
(10) Pub. No.: US 2008/0070865 A1
(43) Pub. Date: Mar. 20, 2008

(54) TRIPHENYLPHOSPHINE OXIDE DERIVATIVES AND USES THEREOF

(75) Inventors: R. KYLE PALMER, Cranbury, NJ (US); Robert W. Bryant, Princeton, NJ (US); Dennis Sprous, Ware, MA (US)

Correspondence Address: STERNE, KESSLER, GOLDSTEIN & FOX P.L.L.C., 1100 NEW YORK AVENUE, N.W., WASHINGTON, DC, 20005, US

(73) Assignee: Redpoint Bio Corporation, Ewing, NJ (US)

(21) Appl. No.: 11/856,518

(22) Filed: Sep. 17, 2007

Related U.S. Application Data
(60) Provisional application No. 60/844,657, filed on Sep. 15, 2006.

(51) Int. Cl. A61K 31/662; A61P 39/00; C07K 14/465; C07K 14/47; C07K 14/705; C12N 5/06; C12N 5/08

(52) U.S. Cl. 514/75; 435/375; 530/350

(57) ABSTRACT

The present invention is directed to the use of a compound having the formula

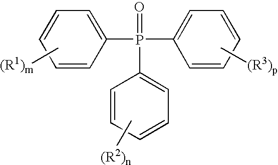

wherein R¹, R², R³, m, n and p are defined herein. The compounds of the present invention are useful as inhibitors of certain taste perceptions and functions. The invention is also directed to compositions comprising a compound according to the above formula.

The application claims the benefit of U.S. Provisional Application No. 60/844,657, filed Sep. 15, 2006, which is herein incorporated by reference in its entirety.

### BACKGROUND OF THE INVENTION

1. Field of the Invention

The present invention relates to the use of compounds of Formula I for inhibiting certain taste functions and perceptions and related uses. The invention is also directed to, among other things, compositions comprising a compound of Formula I that can be used in pharmaceutical, food, and other products to inhibit certain taste functions and perceptions.

2. Background Art

Taste perception plays a critical role in both the nutritional status of human beings and the basic survival of animals. Margolskee, R. F., J. Biol. Chem. 277:1-4 (2002); Avenet, P. and Lindemann, B., J. Membrane Biol. 112:1-8 (1989). The task of taste perception is carried out by taste receptor cells (TRCs). TRCs have the ability to perceive the multitude of compounds that are associated with a given taste and then convert that perception to a signal that is deciphered by the brain, resulting in the sensation of sweet, bitter, sour, salty, or umami (savory) taste.

TRCs are polarized epithelial cells, meaning that they have specialized apical and basolateral membranes. A taste bud contains approximately 60 to 100 TRCs. Each TRC has a portion of its membrane exposed on the mucosal surface of the tongue. Kinnamon, S. C., TINS 11:491-496 (1988). Sensory transduction is initiated by sapid molecules, or “tastants,” that interact with microvillar processes on the apical membrane of TRCs. The tastants bind specific membrane receptors, resulting in a voltage change across the cell membrane. In turn, this depolarizes, or changes the electric potential, of the cell, causing transmitter release and excitation of primary gustatory nerve fibers.

One recently discovered transmembrane protein, TRPM5, has been shown to be essential for taste transduction. Perez et al., Nature Neuroscience 5:1169-1176 (2002); Zhang et al., Cell 112:293-301 (2003). This protein is a member of the transient receptor potential (TRP) family of ion channels, forms a channel through the membrane of the taste receptor cell, and is believed to be activated by stimulation of a receptor pathway coupled to phospholipase C and by IP₃-mediated Ca²⁺ release. The opening of this channel is dependent on a rise in Ca²⁺ levels. Hofmann et al, Current Biol. 13:1153-1158 (2003). The activation of this channel leads to depolarization of the TRC, which in turn leads to transmitter release and excitation of primary gustatory nerve fibers.

Because TRPM5 is a necessary part of the taste-perception machinery, its inhibition prevents an animal from sensing particular tastes. Although taste perception is a vital function, the inhibition of undesirable tastes is beneficial under certain circumstances. For example, many active pharmaceutical ingredients of medicines produce undesirable tastes, such as a bitter taste. Inhibition of the bitter taste produced by the medicine may lead to improved acceptance by the patient.

Traditionally, sweeteners and flavorants have been used to mask the bitter taste of pharmaceuticals. The sweetener or flavorant is known to activate other taste pathways and at sufficiently high concentration this serves to mask the bitter taste of the pharmaceutical. However, this approach has proved ineffective at masking the taste of very bitter compounds. Microencapsulation in a cellulose derivative has also been used to mask the bitter taste of pharmaceuticals. However, this approach prevents rapid oral absorption of the pharmaceutical.

A number of other methods have been suggested to inhibit, alter, or mask unwanted tastes, including the use of 5′-adenosine carboxylic acid (AMP) and 5′-inosine carboxylic acid (IMP) as potential bitterness inhibitors. See U.S. Pat. No. 6,540,978. However, the presently available compounds are lacking in desirable characteristics.

Another aspect of taste is its role in food intake. Studies have shown increased food intake as palatability increased. Sorensen, et al., Int. J. Obes. Relat. Metab. Disord. 27(10):1152-66 (2003). For instance, certain drugs, such as antihypertensives and antihyperlipidemics, have been reported to produce untoward alterations in taste and may result in decreased food intake. Doty, et al., J Hypertens. 21(10):1805-13 (2003). Taste impairment has also been associated with radiation treatments for head and neck cancer and this taste impairment has been considered to be one of the factors associated with reduces appetite and altered patterns of food intake. Vissink, et al., Crit. Rev. Oral Biol. Med. 14(3):213-25 (2003). Decreased food consumption has also been correlated with loss of taste sensations in the elderly. Shiffman, S. S., J. Am. Med. Ass'n 278(16):1357-1362 (1997).

At present, while there are a number of agents that are or have been on the market to reduce appetite and food intake, such as amphetamine derivatives and fenfluramine, many have serious side effects. More selective approaches, e.g., neuro-regulation via peptide mimetics/antagonists, are still in developmental phases.

Therefore, there exists a need for compounds that can effectively inhibit an unwanted taste without exhibiting one or more of the side effects of the prior art taste masking agents.

### SUMMARY OF THE INVENTION

A first aspect of the present invention is directed to a method of inhibiting a taste modulating protein, said method comprising contacting said protein with a compound of Formula I or a physiologically acceptable salt thereof.

An additional aspect of the present invention is directed to a method of inhibiting the depolarization of a taste receptor cell, said method comprising contacting said cell with a compound of Formula I or a physiologically acceptable salt thereof.

An additional aspect of the present invention is directed to a method of inhibiting the taste of a pharmaceutical, comprising administering one or more compounds of Formula I, or a physiologically acceptable salt thereof, in conjunction with the administration of said pharmaceutical to a subject.

An additional aspect of the present invention is directed to a method of inhibiting the taste of a food product, comprising administering one or more compounds of Formula I, or a physiologically acceptable salt thereof, in conjunction with the administration of said pharmaceutical to a subject.

An additional aspect of the present invention is directed to a pharmaceutical composition comprising an active agent, optionally one or more pharmaceutically acceptable carriers, and one or more compounds of Formula I or a physiologically acceptable salt thereof.

An additional aspect of the present invention is directed to a food product comprising one or more compounds according to Formula I or a physiologically acceptable salt thereof.

An additional aspect of the present invention is directed to a method of decreasing the palatability of food and its intake comprising administering one or more compounds of Formula I to a subject in need of such treatment.

These and additional aspects of the present invention are described in detail below.

### BRIEF DESCRIPTION OF THE DRAWINGS/FIGURES

The accompanying drawings, which are incorporated herein and form a part of the specification, serve to explain the principles of the invention and to enable a person skilled in the pertinent art to make and use the invention.

FIG. 1 illustrates the generation of the TRPM5 FLIPR response.

FIGS. 2A and 2B illustrate the TRPM5-dependent fluorescent signal in HEK293 cells, as explained in Example 6.

### DETAILED DESCRIPTION OF THE INVENTION

The present invention provides compounds and compositions that are useful, for example, for inhibiting the activity of a taste modulating protein. Other aspects of the present invention are described in detail herein.

### Methods of Use

A first aspect of the present invention is directed to a method of inhibiting a taste modulating protein, said method comprising contacting said protein with a compound of Formula I:

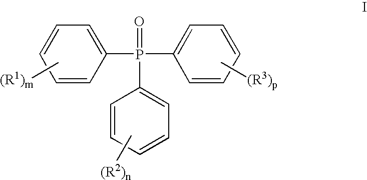

or a physiologically acceptable salt thereof, wherein

R¹, R² and R³ are independently selected from the group consisting of C₁₋₆ alkyl, C₂₋₆ alkenyl, C₂₋₆ alkynyl, hydroxy, C₁₋₆ alkoxy, amino, C₁₋₆ alkylamino, C₁₋₆ dialkylamino, and halo; and

m, n, and p are independently an integer from 0 to 5.

In one embodiment, R¹, R², and R³ are hydrogen. In another embodiment, R¹ and R² are hydrogen. In another embodiment, at least one of R¹, R², and R³ is hydroxy. In another embodiment, R³ is an amino group. In another embodiment, R³ is methoxy. In another embodiment, R³ is diethylamino. Examples of suitable compounds for use in the method of the present invention include:

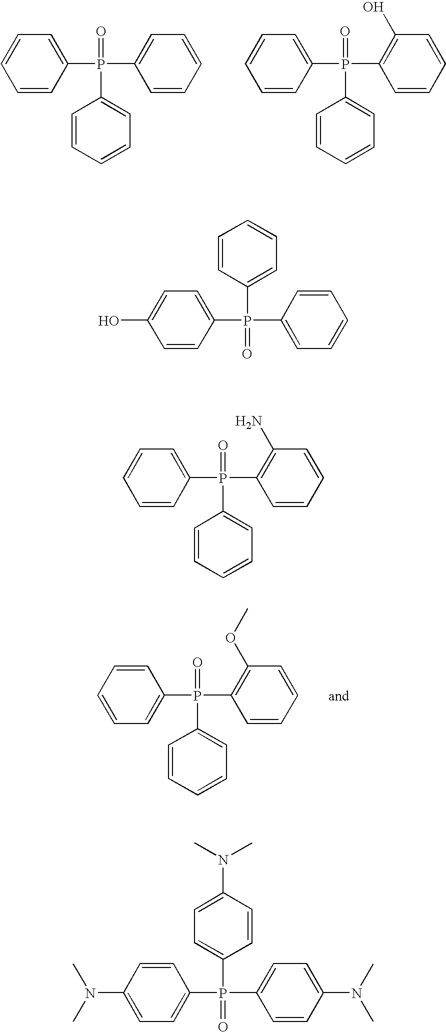

The methods of the present invention also include the use of a physiologically acceptable salt of a compound according to Formula I. The term physiologically acceptable salt refers to an acid- and/or base-addition salt of a compound according to Formula I. Acid-addition salts can be formed by adding an appropriate acid to the compound according to Formula I. Base-addition salts can be formed by adding an appropriate base to the compound according to Formula I. Said acid or base does not substantially degrade, decompose, or destroy said compound according to Formula I. Examples of suitable physiologically acceptable salts include hydrochloride, hydrobromide, acetate, furmate, maleate, oxalate, and succinate salts. Other suitable salts include sodium, potassium, carbonate, and tromethamine salts.

It is also to be understood that the present invention is considered to encompass the use of stereoisomers as well as optical isomers, e.g., mixtures of enantiomers as well as individual enantiomers and diastereomers, which arise as a consequence of structural asymmetry in selected compounds of the present series. It is further understood that the present invention encompasses the use of tautomers of a compound of Formula I. Tautomers are well-known in the art and include keto-enol tautomers.

The compounds of Formula I may also be solvated, including hydrated. Hydration may occur during manufacturing of the compounds or compositions comprising the compounds, or the hydration may occur over time due to the hygroscopic nature of the compounds.

Certain compounds within the scope of Formula I may be derivatives referred to as “prodrugs.” The expression “prodrug” denotes a derivative of a known direct acting agent, wherein the derivative has therapeutic value that may be similar to, greater than, or less than that of the agent. Generally, the prodrug is transformed into the active agent by an enzymatic or chemical process when delivered to the subject, cell, or test media. In certain instances, prodrugs are derivatives of the compounds of the invention which have metabolically cleavable groups and become by solvolysis or under physiological conditions the compounds of the invention which are pharmaceutically active in vivo. For example, ester derivatives of compounds of this invention are often active in vivo, but not in vitro. Other derivatives of the compounds of this invention have activity in both their acid and acid derivative forms, but the acid derivative form often offers advantages of solubility, tissue compatibility, or delayed release in the mammalian organism (See Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with an amine. Simple aliphatic or aromatic esters derived from acidic groups pendent on the compounds of this invention are preferred prodrugs. In some cases, it is desirable to prepare double ester type prodrugs such as (acyloxy) alkyl esters or ((alkoxycarbonyl)oxy)alkyl esters.

When any variable occurs more than one time in any constituent or in Formula I, its definition on each occurrence is independent of its definition at every other occurrence, unless otherwise indicated. Also, combinations of substituents and/or variables are permissible only if such combinations result in stable compounds.

The term “alkyl,” as used herein by itself or as part of another group, refers to both straight and branched chain radicals of up to 10 carbons, unless the chain length is limited thereto, such as methyl, ethyl, propyl, isopropyl, butyl, 1-methylpropyl, 2-methylpropyl, pentyl, 1-methylbutyl, isobutyl, pentyl, t-amyl (CH₃CH₂(CH₃)₂C—), hexyl, isohexyl, heptyl, octyl, or decyl.

The term “alkenyl,” as used herein by itself or as part of another group, refers to a straight or branched chain radical of 2-10 carbon atoms, unless the chain length is limited thereto, including, but not limited to, ethenyl, 1-propenyl, 2-propenyl, 2-methyl-1-propenyl, 1-butenyl, 2-butenyl, 3-butenyl, pentenyl, 1-hexenyl, and 2-hexenyl.

The term “alkynyl,” as used herein by itself or as part of another group, refers to a straight or branched chain radical of 2-10 carbon atoms, unless the chain length is limited thereto, wherein there is at least one triple bond between two of the carbon atoms in the chain, including, but not limited to, ethynyl, 1-propynyl, 2-propynyl, 1-butynyl, 2-butynyl, 1-methyl-2-butynyl, 1-methyl-3-butynyl, 2-methyl-3-pentynyl, hexynyl, and heptynyl.

In instances herein where there is an alkenyl or alkynyl moiety as a substituent group, the unsaturated linkage is preferably not directly attached to a nitrogen, oxygen or sulfur moiety.

The term “alkoxy,” as used herein by itself or as part of another group, refers to any of the above alkyl groups linked to an oxygen atom. Typical examples are methoxy, ethoxy, isopropyloxy, sec-butyloxy, and t-butyloxy.

The term “halogen” or “halo,” as used herein by itself or as part of another group, refers to chlorine, bromine, fluorine or iodine.

The term “alkylamine” or “alkylamino,” as used herein by itself or as part of another group, refers to the group NH₂ wherein one hydrogen has been replaced by an alkyl group, as defined above.

The term “dialkylamine” or “dialkylamino,” as used herein by itself or as part of another group refers to the group, NH₂ wherein both hydrogens have been replaced by alkyl groups, as defined above.

The term “haloalkyl,” as used herein by itself or as part of another group, refers to any of the above alkyl groups wherein one or more hydrogens thereof are substituted by one or more halo moieties. Typical examples include fluoromethyl, trifluoromethyl, trichloroethyl, and trifluoroethyl.

The term “haloalkenyl,” as used herein by itself or as part of another group, refers to any of the above alkenyl groups wherein one or more hydrogens thereof are substituted by one or more halo moieties. Typical examples include fluoroethenyl, difluoroethenyl, and trichloroethenyl.

The term “oxy” means an oxygen (O) atom.

Although detailed definitions have not been provided for every term used above, each term is understood by one of ordinary skill in the art.

As mentioned above, the above described compounds may be used to inhibit a taste modulating protein. Such inhibition may be in vitro or in vivo. The amount of the compound of Formula I, or any of the specific subgroups, subclasses, or specific compounds described above, used to inhibit the taste modulating protein may not necessarily be the same when used in vivo compared to in vitro. Factors such as pharmacokinetics and pharmacodynamics of the particular compound may require that a larger or smaller amount of the compound of Formula I, or any of the specific subgroups, subclasses, or specific compounds described above, be used when inhibiting a taste modulating protein in vivo. Accordingly, one aspect of the present invention is a method of inhibiting a taste modulating protein, comprising contacting the taste modulating protein with a compound according to Formula I, or any of the specific subgroups, subclasses, or specific compounds described above. In one embodiment of this aspect of the present invention, the method comprises contacting a cell with a compound of Formula I, or any of the specific subgroups, or specific compounds described above, wherein said cell expresses said taste modulating protein. In another embodiment of the present invention, the method comprises administering a compound of Formula I, or any of the specific subgroups, or specific compounds described above, to a subject in an amount sufficient to inhibit a taste modulating protein, wherein said subject has or expresses said taste modulating protein. Furthermore, when administered orally, the compound may be dispersed or diluted by saliva.

By way of example, the present invention is directed to a method of inhibiting a taste modulating protein, comprising contacting said protein with a compound of Formula I, or any of the specific subgroups and specific compounds listed above, and inhibiting the protein by at least about 10%, 20%, 30%, 40%, 50%, 60%, 70%, 80%, 90%, or 95%, or from about 50% to about 99%. In another embodiment, the method comprises contacting said protein with a compound of Formula I, or any of the specific subgroups and specific compounds listed above, and inhibiting the protein by about 10% to about 50%. In another embodiment, the present invention is directed to a method of inhibiting a taste modulating protein, comprising contacting said protein with a compound of Formula I, or any of the specific subgroups and specific compounds listed above, and inhibiting the protein by at least about 10%, 20%, 30%, 40%, 50%, 60%, 70%, 80%, 90%, or 95%, or from about 50% to about 99%, or alternatively from about 10% to about 50%, and wherein said taste modulating protein is a naturally occurring taste modulating protein. In another embodiment, the present invention is directed to a method of inhibiting a taste modulating protein, comprising contacting said protein with a compound of Formula I, or any of the specific subgroups or specific compounds listed above, and inhibiting the protein by at least about 10%, 20%, 30%, 40%, 50%, 60%, 70%, 80%, 90%, or 95%, or from about 50% to about 99%, or alternatively from about 10% to about 50%, and wherein said protein is a naturally occurring human taste modulating protein.

Any amount of the compound of Formula I that provides the desired degree of inhibition can be used. For example, a compound of Formula I may be used at a concentration of about 0.1 μM to about 1,000 μM to inhibit a taste modulating protein. Alternatively, concentrations of about 1, 10 or 100 μM of a compound of Formula I may be used to inhibit a taste modulating protein. In certain embodiments, a single dose or two to four divided daily doses, provided on a basis of about 0.001 to 100 mg per kilogram of body weight per day, preferably about 0.01 to about 25 mg/kg of body weight per day is appropriate. The substance is preferably administered orally, but parenteral routes such as the subcutaneous, intramuscular, intravenous or intraperitoneal routes or any other suitable delivery system, such as intranasal or transdermal routes can also be employed.

As used herein, the term “inhibiting” and grammatical variants thereof refers to interfering with the normal activity of. For example, inhibiting a taste modulating protein means interfering with the normal activity of a taste modulating protein. Inhibiting includes but is not necessarily limited to modulating, modifying, inactivating, and the like.

As used herein, the phrase “taste modulating protein” refers to a TRPM5 protein, and includes naturally and recombinantly produced TRPM5 proteins; natural, synthetic, and recombinant biologically active polypeptide fragments of said protein; biologically active polypeptide variants of said protein or fragments thereof, including hybrid fusion proteins and dimers; biologically active polypeptide analogs of said protein or fragments or variants thereof, including cysteine substituted analogs. The taste modulating protein may be a nonhuman protein, for example a nonhuman mammalian protein, or in other embodiments a nonhuman protein such as but not limited to a cow, horse, sheep, pig, chicken, turkey, quail, cat, dog, mouse, rat, rabbit, monkey, or guinea pig taste modulating protein. The taste modulating protein may be generated and/or isolated by any means known in the art. An example of the taste modulating protein and methods of producing the protein are disclosed in, for example, Liu and Liman, Proc. Nat'l Acad. Sci. USA 100: 15160-15165 (2003); D. Prawitt, et al., Proc. Nat'l Acad. Sci. USA 100:15166-71 (2003); and Ulrich, N. D., et al., Cell Calcium 37: 267-278 (2005); each of which is fully incorporated by reference herein.

A homologue is a protein that may include one or more amino acid substitutions, deletions, or additions, either from natural mutations of human manipulation. Thus, by way of example, a taste modulating protein may include one or more amino acid substitutions, deletions or additions, either from natural mutations or human manipulation. As indicated, changes are preferably of a minor nature, such as conservative amino acid substitutions that do not significantly affect the folding or activity of the protein.

The variant taste modulating proteins which may be inhibited in accordance with the present invention comprise non-conservative modifications (e.g., substitutions). By “nonconservative” modification herein is meant a modification in which the wild-type residue and the mutant residue differ significantly in one or more physical properties, including hydrophobicity, charge, size, and shape. For example, modifications from a polar residue to a nonpolar residue or vice-versa, modifications from positively charged residues to negatively charged residues or vice versa, and modifications from large residues to small residues or vice versa are nonconservative modifications. For example, substitutions may be made which more significantly affect: the structure of the polypeptide backbone in the area of the alteration, for example the alpha-helical or beta-sheet structure; the charge or hydrophobicity of the molecule at the target site; or the bulk of the side chain. The substitutions which in general are expected to produce the greatest changes in the polypeptide's properties are those in which (a) a hydrophilic residue, e.g., seryl or threonyl, is substituted for (or by) a hydrophobic residue, e.g., leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine. In one embodiment, the variant taste modulating proteins used in accordance with the present invention have at least one nonconservative modification.

In other embodiments, the method of the invention comprises inhibiting a taste modulating protein that is a nonhuman protein, such as but not limited to a cow, horse, sheep, pig, chicken, turkey, quail, cat, dog, mouse, rat, rabbit, monkey, or guinea pig taste modulating protein.

An additional aspect of the present invention is a method of inhibiting the depolarization of a taste receptor cell, comprising contacting the taste receptor cell with a compound according to Formula I, or any of the specific subgroups, or specific compounds described above. For example, a compound of Formula I may inhibit the depolarization of a taste receptor cell by a mechanism other than, or in addition to, the mechanism of inhibiting a taste receptor protein. In one embodiment of this aspect of the present invention, the method comprises contacting a taste receptor cell with a compound of Formula I, or any of the specific subgroups, or specific compounds described above, wherein said taste receptor cell can detect a sweet, bitter, sour, salty, or umami taste. In another embodiment of the present invention, the method comprises administering a compound of Formula I, or any of the specific subgroups, or specific compounds described above, to a subject in an amount sufficient to inhibit the depolarization of a taste receptor cell. Furthermore, when administered orally, the compound may be dispersed or diluted by saliva.

By way of example, the present invention is directed to a method of inhibiting the depolarization of a taste receptor cell, comprising contacting said taste receptor cell with a compound of Formula I, or any of the specific subgroups and specific compounds listed above, and inhibiting the depolarization of the taste receptor cell by at least about 10%, 20%, 30%, 40%, 50%, 60%, 70%, 80%, 90%, or 95%, or from about 60% to about 99%, or alternatively from about 30% to about 75%. In another embodiment, the present invention is directed to a method of inhibiting the depolarization of a taste receptor cell, comprising contacting said protein with a compound of Formula I, or any of the specific subgroups and specific compounds listed above, and inhibiting the depolarization of the taste receptor cell by at least about 10%, 20%, 30%, 40%, 50%, 60%, 70%, 80%, 90%, or 95%, or from about 50% to about 99%, or alternatively from about 20% to about 60%, and wherein said taste receptor cell is a naturally occurring taste modulating protein. In another embodiment, the present invention is directed to a method of inhibiting a taste receptor cell, comprising contacting said protein with a compound of Formula I, or any of the specific subgroups or specific compounds listed above, and inhibiting the taste receptor cell by at least about 10%, 20%, 30%, 40%, 50%, 60%, 70%, 80%, 90%, or 95%, or from about 50% to about 99%, or alternatively from about 40% to about 80%, and wherein said taste receptor cell is a human taste receptor cell.

Any amount of the compound of Formula I that provides the desired degree of inhibition can be used. For example, a compound of Formula I may be used at a concentration of about 0.1 μM to about 1,000 μM to inhibit a taste receptor cell. Alternatively, concentrations of about 1 μM, 50 μM, or 100 μM of a compound of Formula I may be used to inhibit the depolarization of a taste receptor cell.

In certain embodiments, a single dose or two to four divided daily doses, provided on a basis of about 0.001 to 100 mg per kilogram of body weight per day, preferably about 0.01 to about 25 mg/kg of body weight per day is appropriate. When inhibiting a taste receptor cell in vivo, the compound of Formula I is preferably administered orally.

In one embodiment of this aspect of the present invention, the method comprises contacting a taste receptor cell with a compound of Formula I, or any of the specific subgroups, or specific compounds described above, wherein said taste receptor cell can detect a sweet, bitter, sour, salty, or umami taste. In another embodiment of the present invention, the method comprises administering a compound of Formula I, or any of the specific subgroups, or specific compounds described above, to a subject in an amount sufficient to inhibit the depolarization of a taste receptor cell. Furthermore, when administered orally, the compound may be dispersed or diluted by saliva.

In another embodiment, a compound according to Formula I, or any of the specific subgroups, or specific compounds described above, is useful for inhibiting a taste, such as an undesirable taste of a food product. Examples of food products having an undesirable taste include, but are not necessarily limited to, citrus fruits such as grapefruit, orange, and lemon; vegetables such as tomato, pimento, celery, melon, carrot, potato and asparagus; seasoning or flavoring materials, such as soy sauce and red pepper; soybean products; fish products; meats and processed meats; dairy products such as cheese; breads and cakes; and confectioneries such as candies, chewing gum and chocolate. Other examples of food products envisioned in accordance with the present invention are described below and throughout the specification.

The method may be performed such that the taste of the food product being inhibited by the compound of Formula I is inhibited by at least about 10%, 20%, 30%, 40%, 50%, 60%, 70%, 80%, 90%, or 95%, or from about 60% to about 99%, or alternatively from about 20% to about 50%. Thus, in a more specific embodiment, the method comprises administering a food product comprising one or more food ingredients and one or more compounds according to Formula I, wherein the one or more compounds according to Formula I are present in an amount sufficient to inhibit a bitter taste, produced by the food product, by at least about 10%, 20%, 30%, 40%, 50%, 60%, 70%, 80%, 90%, or 95%, or from about 60% to about 99%, or alternatively from about 30% to about 70%. Of course, in other embodiments, a taste may be inhibited to differing extents.

Any amount of the compound of Formula I that provides the desired degree of taste inhibiting can be used. For example, a compound of Formula I may be used at a concentration of about 0.1 μM to about 5,000 μM to inhibit a bitter taste. Alternatively, concentrations of about 1 μM, 100 μM, or 500 μM of a compound of Formula I may be used to inhibit a sweet taste.

A food product may also include beverages and drinks. Examples of drinks having an undesirable or unwanted taste include, but are not limited to, juices of citrus fruits and vegetables, soybean, milk, coffee, cocoa, black tea, green tea, fermented tea, semi-fermented tea, refreshing drinks, beverages and milk. In certain embodiments, the taste inhibiting effective amount of a compound according to Formula I, or any of the specific subgroups, or specific compounds described above, has a range of from about 0.01 to about 5.0 grams per 100 mL. In other embodiments, the taste inhibiting effective amount of a compound according to Formula I, or any of the specific subgroups, or specific compounds described above, has a range of from about 0.5 to about 2 grams per 100 mL. Alternatively, a compound according to Formula I, or any of the specific subgroups, subclasses, or specific compounds described above, is administered in an amount of about 1 gram per 100 mL.

The method of the present invention in its various embodiments may be used to inhibit one or more tastes selected from the group consisting of sweet, bitter, sour, salty, or umami. Preferably, the method of the present invention inhibits a bitter and/or sweet taste.

As used herein, the phrase “inhibit a taste” and grammatical variants thereof, such as “taste inhibiting” and “inhibiting a taste,” refers to interfering with the perception of a taste. The taste may be sensed to a lesser degree or not sensed at all by application of the present invention.

An additional aspect of the present invention is a method of inhibiting a taste of a pharmaceutical composition, comprising administering a compound according to Formula I, or any of the specific subgroups, or specific compounds described above, to a subject receiving the pharmaceutical composition. The compound of Formula I may be administered together with the pharmaceutical composition as separate compositions, for example either concurrently or sequentially. The compound of Formula I may administered, or caused to be administered, prior to the pharmaceutical agent producing the taste to be inhibited. Alternatively, the compound for Formula I may be administered as a component of the pharmaceutical composition.

By way of example, the method may be performed such that the taste being inhibited by the compound of Formula I is inhibited by at least about 10%, 20%, 30%, 40%, 50%, 60%, 70%, 80%, 90%, or 95%, or from about 60% to about 99%, or alternatively from about 25% to about 50%. Thus, in a more specific embodiment, the method comprises administering a pharmaceutical composition comprising a pharmaceutically active agent, optionally one or more excipients, and one or more compounds according to Formula I, wherein the one or more compounds according to Formula I are present in an amount sufficient to inhibit a bitter taste, produced by the pharmaceutically active agent, by at least about 10%, 20%, 30%, 40%, 50%, 60%, 70%, 80%, 90%, or 95%, or from about 60% to about 99%, or alternatively from about 30% to about 60%. In another embodiment, the compound of Formula I is administered in a ratio of from about 10:1 to about 1:10 in relation to the pharmaceutical agent.

By way of additional examples, the method of inhibiting a taste of a pharmaceutical composition may comprise inhibiting a taste produced by one or more agents selected from the group consisting of antipyretics, analgesics, laxatives, appetite depressants, antacidics, antiasthmatics, antidiuretics, agents active against flatulence, antimigraine agents, psychopharmacological agents, spasmolytics, sedatives, antihyperkinetics, tranquilizers, antihistaminics, decongestants, beta-receptor blockers, agents for alcohol withdrawal, antitussives, fluorine supplements, local antibiotics, corticosteroid supplements, agents against goiter formation, antiepileptics, agents against dehydration, antiseptics, NSAIDs, gastrointestinal active agents, alkaloids, supplements for trace elements, ion-exchange resins, cholesterol-depressant agents, lipid-lowering agents, antiarrhythmics, and expectorants. Further specific examples of pharmaceutical compostions in accordance with the method of the invention are described below.

Additionally, the method of inhibiting a taste of a pharmaceutical composition may comprise inhibiting a taste produced by a counterterrorism pharmaceutical. Because of the increased risk of terrorist attacks, such as chemical, nuclear, or biological attacks, the use of counterterrorism pharmaceutical agents is expected to increase in the future. A counterterrorism pharmaceutical agent includes those pharmaceutical agents that are useful in counteracting agents that can be used in a terrorist attack. Agents that have been used in terrorist acts, or considered as useful for carrying out future terrorist acts, include ricin, sarin, radioactive agents and materials, and anthrax. Pharmaceutical agents that counteract these agents are useful as a counterterrorism pharmaceutical. Such counterterrorism pharmaceuticals include, but are not limited to, antiobiotics such as ciprofloxacin and doxycycline; potassium iodide; and antiviral agents. Thus, in one embodiment of the present invention, the method may be performed such that the taste of a counterterrorism pharmaceutical, such as an antibiotic such as ciprofloxacin and doxycycline; potassium iodide; or an antiviral agent, is inhibited by the compound of Formula I by at least about 10%, 20%, 30%, 40%, 50%, 60%, 70%, 80%, 90%, or 95%, or from about 60% to about 99%, or alternatively from about 25% to about 50%. In another embodiment, the compound of Formula I is administered in a ratio of from about 10:1 to about 1:10 in relation to the counterterrorism agent.

In another embodiment, a compound according to Formula I, or any of the specific subgroups, or specific compounds described above, is useful for inhibiting an undesirable taste of a nutriceutical composition. Examples of nutriceutical compositions having an undesirable taste include, but are not necessarily limited to, enteral nutrition products for treatment of nutritional deficit, trauma, surgery, Crohn's disease, renal disease, hypertension, obesity and the like, to promote athletic performance, muscle enhancement or general well being or inborn errors of metabolism such as phenylketonuria. In particular, such nutriceutical formulations may contain one or more amino acids which have a bitter or metallic taste or aftertaste. Such amino acids include, but are not limited to, an essential amino acids selected from the group consisting of L isomers of leucine, isoleucine, histidine, lysine, methionine, phenylalanine, threonine, tryptophan, tyrosine, and valine. Further specific examples of nutraceutical compostions in accordance with the method of the invention are described below.

By way of example, the method may be performed such that the taste being inhibited by the compound of Formula I is inhibited by at least about 10%, 20%, 30%, 40%, 50%, 60%, 70%, 80%, 90%, or 95%, or from about 60% to about 99%, or alternatively from about 20% to about 50% Thus, in a more specific embodiment, the method comprises administering a nutraceutical composition comprising a nutraceutical agent, optionally one or more excipients, and one or more compounds according to Formula I, wherein the one or more compounds according to Formula I are present in an amount sufficient to inhibit a undesired taste, produced by the nutraceutical agent, by at least about 10%, 20%, 30%, 40%, 50%, 60%, 70%, 80%, 90%, or 95%, or from about 60% to about 99%, or alternatively from about 10% to about 50%.

A compound according to Formula I may be incorporated into medical and/or dental compositions. Certain compositions used in diagnostic procedures have an unpleasant taste, such as contrast materials and local oral anesthetics. The inhibitors of the invention may be used to improve the comfort of subjects undergoing such procedures by improving the taste of compositions. In addition, the inhibitors of the invention may be incorporated into pharmaceutical compositions, including tablets and liquids, to improve their flavor and improve patient compliance particularly where the patient is a child or a non-human animal).

In another embodiment, a compound according to Formula I, or any of the specific subgroups, or specific compounds described above, is used to inhibit a taste of a cosmetic product. For example, but not by way of limitation, a compound according to Formula I may be incorporated into face creams, lipsticks, lipgloss, and the like. Also, a compound according to Formula I, or any of the specific subgroups, or specific compounds described above, can be used to inhibit an unpleasant taste of lipbalm, such as Chapstick® or Burt's Beeswax® Lip Balm.

In addition, a compound according to Formula I, or any of the specific subgroups, or specific compounds described above, may be incorporated into compositions that are not traditional foods, pharmaceuticals, or cosmetics, but which may contact taste membranes. Examples include, but are not limited to, soaps, shampoos, toothpaste, denture adhesive, and glue on the surfaces of stamps and envelopes. Thus, the present invention also covers a process of preparing a composition that is not a traditional food, pharmaceutical, or cosmetic, but which may contact taste membranes, according to conventional methods, wherein the improvement comprises adding a compound of Formula I to said composition.

In another embodiment, a compound according to Formula I, or any of the specific subgroups, or specific compounds described above, is used to inhibit a bitter taste associated with one or more the following: bitter pharmaceutical alkaloids such as acetaminophen, ampicillin, chlorpheniramine, chlarithromycin, doxylamine, guaifenesin, ibuprofen, pseudoephidrine hydrochloride, and ranitidine, bitter pharmaceutical metallic salts such as zinc containing bioadhesives (denture adhesive), bitter vitamins, bitter components of foods such as creatine, limonin, naringin, quinizolate, and bitter components of beverages such as caffeine, and humulone. In one embodiment, the concentration of the compound according to Formula I used is in the range of 0.01 mM to 20 mM and may vary depending on the amount of bitter compound used and its bitterness.

In another embodiment, the present invention is directed to a method of inhibiting the taste of a veterinary product, such as veterinary medicines, veterinary food products, veterinary supplements, and the like, that are administered to domesticated animals. In a preferred embodiment, a compound according to Formula I, or any of the specific subgroups, subclasses, or specific compounds described above, is used to inhibit a taste of a veterinary product administered to a cat or dog.

In one embodiment, in each of the methods of inhibiting a taste described herein, a compound according to Formula I, or any of the specific subgroups, or specific compounds described above, is administered in an amount effective to inhibit said taste. As a nonlimiting example, the taste inhibiting effective amount of a compound according to Formula I, or any of the specific subgroups, or specific compounds described above, administered in one embodiment is from about 0.01 to about 5.0 grams per 100 mL.

In other embodiments, in the taste inhibiting methods described herein, a compound according to Formula I, or any of the specific subgroups, or specific compounds described above, is administered in an amount that is sufficient, in combination with the administration of one or more additional taste inhibiting agents, to inhibit said taste. For example, in a method of inhibiting the bitter taste of a liquid pharmaceutical composition, the composition comprises a compound according to Formula I and another taste inhibiting agent, wherein the amount of the compound of Formula I is about 25% to about 75% of the amount required to inhibit the bitter taste in the absence of the other taste inhibiting agent.

In another embodiment, the present invention is directed to a method of decreasing the palatability and/or intake of food, comprising administering to a subject in need of such treatment one or more compounds according to Formula I, or any of the specific subgroups, or specific compounds described above, in an amount sufficient to decrease the palatability and/or intake of food. Taste modulating protein knockout mice have been shown to have diminished taste preference for sucrose, artificial sweeteners, and umami flavors and diminished taste aversion to bitter solutions. See Zhang et al., Cell 112:293-301 (2003). Thus, according to the present invention, a compound according to Formula I, or any of the specific subgroups, or specific compounds described above, may be administered to a subject so that the palatability of food, as experienced by said subject, is decreased. Without being bound by theory, it is believed that a lower palatability of food can lead to a lower intake of food by the subject. Thus, in certain embodiments, by administering a compound according to Formula I, or any of the specific subgroups, or specific compounds described above, to a subject, the subject will consume a decreased amount of food compared to the subject's food intake when not being administered a compound of Formula I, or any of the specific subgroups, or specific compounds described above. In other embodiments, by administering a compound according to Formula I, or any of the specific subgroups, or specific compounds described above, to a subject, the subject will have a lower caloric intake compared to the subject's caloric intake when not being administered a compound of Formula I, or any of the specific subgroups, or specific compounds described above. In other embodiments, administering a compound according to Formula I, or any of the specific subgroups, or specific compounds described above, to a subject can be a dieting means to facilitate or aid weight loss.

In each of the embodiments of methods described above, the subject of the method, unless otherwise limited to, may be any animal which is need of the particular treatment or effect of the method. Such animals include but are not limited to a cow, horse, sheep, pig, chicken, turkey, quail, cat, dog, mouse, rat, rabbit, monkey, or guinea pig taste modulating protein. In other embodiments, the animal is a livestock animal, a domesticated animal, or an animal kept as a pet. In particular embodiments, the subject of the claimed method is a human.

Furthermore, in each of the embodiments of the methods described herein, a compound of Formula I may be used in varying ratios to the agent that is believed to cause the unwanted taste, such as a bitter or sweet taste. For example, a compound of Formula I may be administered in a molar ratio of about 1000:1 to about 1:1000, or alternatively administered in a molar ratio of about 500:1, about 200:1, about 10:1, about 1:1, about 1:10, about 1:200, or about 1:500, relative to the agent that is believed to cause the unwanted taste. In another example, the present invention is directed to a method of inhibiting a bitter taste of a pharmaceutical composition, comprising administering to a subject in need of such method a pharmaceutical composition and a compound according to Formula I, wherein the pharmaceutical composition comprises a pharmaceutically active agent and optionally one or more excipients, and wherein the compound according to Formula I is administered as either a component of the pharmaceutical composition or as a separate dosage form, and wherein molar ratio of the compound of Formula I to the pharmaceutically active agent about 1000:1 to about 1:1000, or alternatively administered in a molar ratio of about 500:1, about 200:1, about 10:1, about 1:1, about 1:10, about 1:200, or about 1:500. As will be appreciated, the various ranges and amounts of the compound of Formula I can be used, with modifications if preferred, in each of the embodiments described herein.

### Compositions

The present invention is also directed to various, useful compositions comprising a compound of Formula I or a physiologically acceptable salt thereof.

In one aspect, the present invention is directed to a pharmaceutical composition comprising a compound of Formula I, as defined above, including any of the specific embodiments, or species described above, and one or more pharmaceutically acceptable carriers. Preferred compositions of the present invention are pharmaceutical compositions comprising a compound selected from one or more embodiments listed above, and one or more pharmaceutically acceptable excipients. Pharmaceutical compositions that comprise one or more compounds of Formula I, or any of the specific subgroups, or specific compounds described above, may be used to formulate pharmaceutical drugs containing one or more active agents that exert a biological effect other than taste inhibition and/or inhibition of a taste modulating protein.

The pharmaceutical composition preferably further comprises one or more active agents that exert a biological effect. Such active agents includes pharmaceutical and biological agents that have an activity other than taste inhibition. Such active agents are well known in the art. See, e.g., The Physician's Desk Reference. Such compositions can be prepared according to procedures known in the art, for example, as described in Remington's Pharmaceutical Sciences, Mack Publishing Co., Easton, Pa., USA. In one embodiment, such an active agent includes bronchodilators, anorexiants, antihistamines, nutritional supplements, laxatives, analgesics, anesthetics, antacids, H₂-receptor antagonists, anticholinergics, antidiarrheals, demulcents, antitussives, antinauseants, antimicrobials, antibacterials, antifungals, antivirals, expectorants, anti-inflammatory agents, antipyretics, and mixtures thereof. The pharmaceutical composition according to the present invention may comprise one or more compounds according to Formula I, as described above, or any of the specific subgroups, or specific compounds described above; an active agent that has a bitter taste; and optionally one or more pharmaceutically acceptable carriers.

In another embodiment, the active agent is selected from the group consisting of antipyretics and analgesics, e.g., ibuprofen, acetaminophen, or aspirin; laxatives, e.g., phenolphthalein dioctyl sodium sulfosuccinate; appetite depressants, e.g., amphetamines, phenylpropanolamine, phenylpropanolamine hydrochloride, or caffeine; antacidics, e.g., calcium carbonate; antiasthmatics, e.g., theophylline; antidiuretics, e.g., diphenoxylate hydrochloride; agents active against flatulence, e.g., simethecon; migraine agents, e.g., ergotaminetartrate; psychopharmacological agents, e.g., haloperidol; spasmolytics or sedatives, e.g., phenobarbitol; antihyperkinetics, e.g., methyldopa or methylphenidate; tranquilizers, e.g., benzodiazepines, hydroxinmeprobramates or phenothiazines; antihistaminics, e.g., astemizol, chloropheniramine maleate, pyridamine maleate, doxlamine succinate, bromopheniramine maleate, phenyltoloxamine citrate, chlorocyclizine hydrochloride, pheniramine maleate, and phenindamine tartrate; decongestants, e.g., phenylpropanolamine hydrochloride, phenylephrine hydrochloride, pseudoephidrine hydrochloride, pseudoephidrine sulfate, phenylpropanolamine bitartrate, and ephedrine; beta-receptor blockers, e.g., propanolol; agents for alcohol withdrawal, e.g., disulfuram; antitussives, e.g., benzocaine, dextromethorphan, dextromethorphan hydrobromide, noscapine, carbetapentane citrate, and chlophedianol hydrochloride; fluorine supplements, e.g., sodium fluoride; local antibiotics, e.g., tetracycline or cleocine; corticosteroid supplements, e.g., prednisone or prednisolone; agents against goiter formation, e.g., colchicine or allopurinol; antiepileptics, e.g., phenyloine sodium; agents against dehydration, e.g., electrolyte supplements; antiseptics, e.g., cetylpyridinium chloride; NSAIDs, e.g., acetaminophen, ibuprofen, naproxen, or salts thereof; gastrointestinal active agents, e.g., loperamide and famotidine; various alkaloids, e.g., codeine phosphate, codeine sulfate, or morphine; supplements for trace elements, e.g., sodium chloride, zinc chloride, calcium carbonate, magnesium oxide, and other alkali metal salts and alkali earth metal salts; vitamins; ion-exchange resins, e.g., cholestyramine; cholesterol-depressant and lipid-lowering substances; antiarrhythmics, e.g., N-acetylprocainamide; and expectorants, e.g., guaifenesin.

Active substances which have a particularly unpleasant taste include antibacterial agents such as ciprofloxacin, ofloxacin, and pefloxacin; antiepileptics such as zonisamide; macrolide antibiotics such as erythromycin; beta-lactam antibiotics such as penicillins and cephalosporins; psychotropic active substances such as chlorpromazine; active substances such as sulpyrine; and agents active against ulcers, such as cimetidine.

In another embodiment, the pharmaceutical composition comprises one or more compounds according to Formula I, or any of the specific subgroups, or specific compounds described above, and at least one amino acid selected from the group consisting of glycine, L-alanine, L-arginine, L-aspartic acid, L-cystine, L-glutamic acid, L-glutamine, L-histidine, L-isoleucine, L-leucine, L-lysine, L-methionine, L-ornithine, L-phenylalanine, L-proline, L-serine, L-threonine, L-tryptophan, L-tyrosine, L-valine, creatine, and mixtures thereof.

In another embodiment, the pharmaceutical composition comprises one or more compounds according to Formula I, or any of the specific subgroups, or specific compounds described above; a biologically active agent that exhibits an activity other than taste inhibition; and at least one amino acid, such as one selected from the group consisting of glycine, L-alanine, L-arginine, L-aspartic acid, L-cystine, L-glutamic acid, L-glutamine, L-histidine, L-isoleucine, L-leucine, L-lysine, L-methionine, L-ornithine, L-phenylalanine, L-proline, L-serine, L-threonine, L-tryptophan, L-tyrosine, L-valine, creatine, and mixtures thereof.

The pharmaceutical compositions of the present invention can be in any form suitable to achieve their intended purpose. Preferably, however, the composition is one which can be administered buccally or orally. Alternatively, the pharmaceutical composition may be an oral or nasal spray.

The pharmaceutical compositions of the invention can be in any form suitable for administration to any animal that can experience the beneficial effects of one or more compounds according to Formula I, or any of the specific subgroups, or specific compounds described above. Foremost among such animals are humans, although the invention is not intended to be so limited. Other suitable animals include canines, felines, dogs, cats, livestock, horses, cattle, sheep, and the like. A veterinary composition, as used herein, refers to a pharmaceutical composition that suitable for non-human animals. Such veterinary compositions are known in the art.

The pharmaceutical preparations of the present invention can be manufactured using known methods, for example, by means of conventional mixing, granulating, dragée-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Pharmaceutical excipients are well known in the art. Suitable excipients include fillers such as saccharides, for example, lactose or sucrose, mannitol or sorbitol, cellulose preparations and/or calcium phosphates, for example, tricalcium phosphate or calcium hydrogen phosphate, as well as binders, such as, starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents can be added, such as, the above-mentioned starches and also carboxymethyl-starch, cross-linked polyvinyl pyrrolidone, agar, or alginic acid or a salt thereof, such as, sodium alginate. Auxiliaries are, above all, flow-regulating agents and lubricants, for example, silica, talc, stearic acid or salts thereof, such as, magnesium stearate or calcium stearate, and/or polyethylene glycol. Dragée cores are provided with suitable coatings that, if desired, are resistant to gastric juices. For this purpose, concentrated saccharide solutions can be used, which may optionally contain gum arabic, talc, polyvinyl pyrrolidone, polyethylene glycol, and/or titanium dioxide, lacquer solutions and suitable organic solvents or solvent mixtures. In order to produce coatings resistant to gastric juices, solutions of suitable cellulose preparations, such as, acetylcellulose phthalate or hydroxypropylmethyl-cellulose phthalate, are used. Dye stuffs or pigments can be added to the tablets or dragee coatings, for example, for identification or in order to characterize combinations of active compound doses.

Liquid dosage forms for oral administration include pharmaceutically acceptable emulsions, solutions, suspensions, syrups, and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

In a further embodiment, the invention is directed to a chewable tablet comprising one or more compounds according to Formula I and one or more biologically active agents. Chewable tablets are known in the art. See, e.g., U.S. Pat. Nos. 4,684,534 and 6,060,078, each of which is incorporated by reference in its entirety. Any kind of medicament may be contained in the chewable tablet, preferably a medicament of bitter taste, natural plant extracts or other organic compounds. More preferably, vitamins such as vitamin A, vitamin B, vitamin B₁, vitamin B₂, vitamin B₆, vitamin C, vitamin E and vitamin K; natural plant extracts such as Sohgunjung-tang extracts, Sipchundaebo-tang extracts and Eleutherococcus senticosus extracts; organic compounds such as dimenhydrinate, meclazine, acetaminophen, aspirin, phenylpropanolamine, and cetylpyridinium chloride; or gastrointestinal agents such as dried aluminum hydroxide gel, domperidone, soluble azulene, L-glutamine and hydrotalcite may be contained in the core.

In another embodiment, the present invention is directed to an orally disintegrating composition wherein said orally disintegrating composition further comprises one or more compounds according to Formula I, or any of the specific subgroups, or specific compounds described above. Orally disintegrating tablets are known in the art. See, e.g., U.S. Pat. Nos. 6,368,625 and 6,316,029, each of which is hereby incorporated by reference in its entirety.

In another embodiment, the present invention is further directed to a nasal composition further comprising one or more compounds according to Formula I, or any of the specific subgroups, or specific compounds described above. Nasal sprays are known in the art. See, e.g., U.S. Pat. No. 6,187,332. Addition of one or more compounds according to Formula I to a nasal spray can reduce the experience of an unpleasant taste associated with the composition of the nasal spray. By way of a nonlimiting example, a nasal spray composition according to the present invention comprises water (such as 95-98 weight percent), a citrate (such as 0.02 M citrate anion to 0.06 M citrate anion), a compound according to Formula I, and optionally phosphate (such as 0.03 M phosphate to 0.09 M phosphate).

In another embodiment, the present invention is directed to a solid dosage form comprising a water and/or saliva activated effervescent granule, such as one having a controllable rate of effervescence, and a compound according to Formula I, or any of the specific subgroups, or specific compounds described above. The effervescent composition may further comprise a pharmaceutically active compound. Effervescent pharmaceutical compositions are known in the art. See, e.g., U.S. Pat. No. 6,649,186, which is incorporated by reference in its entirety. The effervescent composition can be used in pharmaceutical, veterinary, horticultural, household, food, culinary, pesticidal, agricultural, cosmetic, herbicidal, industrial, cleansing, confectionery and flavoring applications. Formulations incorporating the effervescent composition comprising a compound according to Formula I can further include one or more additional adjuvants and/or active ingredients which can be chosen from those known in the art including flavors, diluents, colors, binders, filler, surfactant, disintegrant, stabilizer, compaction vehicles, and non-effervescent disintegrants.

In another embodiment, the present invention is directed to a film-shaped or wafer-shaped pharmaceutical composition that comprises a compound according to Formula I, or any of the specific subgroups, or specific compounds described above, and is capable of disintegrating. Such a film-shaped or wafer-shaped pharmaceutical composition can be configured, for example, as quickly disintegrating administration forms, e.g., administration forms disintegrating within a period of 1 second up to 3 minutes, or as slowly disintegrating administration forms, e.g., administration forms disintegrating within a period of 3 to 15 minutes.

The indicated disintegration times can be set to the above-mentioned ranges by using, for example, matrix-forming polymers which have different disintegrating, or solubility, characteristics. Thus, by mixing the corresponding polymer components, the disintegration time can be adjusted. In addition, disintegrants are known which “draw” water into the matrix and cause the matrix to burst open from within. As a consequence, certain embodiments of the invention include such disintegrants for the purpose of adjusting the disintegration time.

Suitable are polymers for use in the film-shaped or wafer-shaped pharmaceutical composition include cellulose derivatives, polyvinyl alcohol (e.g. MOWIOL™), polyacrylates, polyvinyl pyrrolidone, cellulose ethers, such as ethyl cellulose, as well as polyvinyl alcohol, polyurethane, polymethacrylates, polymethyl methacrylates and derivatives and copolymerisates of the aforementioned polymers.

In certain embodiments, the total thickness of the film-shaped or wafer-shaped pharmaceutical composition according to the invention is preferably 5 μm up to 10 mm, preferably 30 μm to 2 mm, and with particular preference 0.1 mm to 1 mm. The pharmaceutical preparations may round, oval, elliptic, triangular, quadrangular or polygonal shape, but they may also have any rounded shape.

In another embodiment, the present invention is directed to a composition comprising a medicament or agent contained in a coating that surrounds a gum base formulation and further comprising a taste-inhibiting amount of a compound according to Formula I, or any of the specific subgroups, or specific compounds described above. Preferably, the coating comprises at least 50% by weight of the entire product. As the center is chewed, the medicament or agent is released into the saliva. For example, U.S. Pat. No. 6,773,716, which is incorporated herein by reference in its entirety, discloses a suitable medicament or agent contained in a coating that surrounds a gum base formulation. One or more compounds according to Formula I, or any of the specific subgroups, subclasses, or specific compounds described above, can be used in preparing the coating. Optionally, the composition may further comprise high-intensity sweeteners and appropriate flavors. It has been found that with respect to certain medicaments or agents that may have an astringent or bitter taste that by adding a inhibiting agent to the formulation, that a much more palatable formulation, including the medicament, can be provided. In this regard, even though the medicament in, for example, its powder form may be bitter or have an offensive taste, the matrix used as the coating of the present invention, including the inhibiting agent, will afford a product having acceptable medicinal properties. The compound according to Formula I, or any of the specific subgroups, or specific compounds described above, may be present in varying amounts, such as about 30% 50%, 75%, or 90%. In another embodiment, the compound according to Formula I may be present in about 30% to about 99%. In other embodiments, the compound according to Formula I is present in about 1% to about 30%.

In yet another embodiment, the present invention is directed to a process of preparing an improved composition comprising a medicament or agent contained in a coating that surrounds a gum base formulation, wherein the improvement comprises adding a compound according to Formula I, or any of the specific subgroups, or specific compounds described above, to the coating that surrounds the gum base formulation. The compound according to Formula I may be added in varying amounts, such as about 30% 50%, 75%, 80%, or 90%, or from about 10% to about 90%. In other embodiments, the compound according to Formula I is present in about 1% to about 30%.

In a further embodiment, the invention is directed to a pharmaceutical composition suitable for aerosol administration, comprising a compound according to Formula I, or any of the specific subgroups, or specific compounds described above, and a suitable carrier. The aerosol composition may further comprises pharmaceutically active agent. Aerosol compositions are known in the art. See, e.g., U.S. Pat. No. 5,011,678, which is hereby incorporated by reference in its entirety. As a nonlimiting example, an aerosol composition according to the present invention may comprise a medically effective amount of a pharmaceutically active substance, one or more compounds according to Formula I, or any of the specific subgroups, or specific compounds described above, and a biocompatible propellant, such as a (hydro/fluoro)carbon propellant.

In certain embodiments, the pharmaceutical compositions of the invention comprise from about 0.001 mg to about 1000 mg of a compound of Formula I, or any of the specific subgroups, or specific compounds described above. In another embodiment, the compositions of the invention comprise from about 0.01 mg to about 10 mg of a compound of Formula I, or any of the specific subgroups, or specific compounds described above.

In another embodiment, the composition of the invention comprises a compound of Formula I, or any of the specific subgroups, or specific compounds described above, in an amount sufficient to inhibit a taste modulating protein. By way of example, the present invention is pharmaceutical or veterinary composition, comprising a compound of Formula I, or any of the specific subgroups and specific compounds listed above, in an amount sufficient to a taste modulating protein by at least about 10%, 20%, 30%, 40%, 50%, 60%, 70%, 80%, 90%, or 95%, or from about 50% to about 99%, or alternatively from about 10% to about 40%. In another embodiment, the present invention is directed to a method of inhibiting a taste modulating protein, comprising contacting said taste modulating protein with a compound of Formula I, or any of the specific subgroups and specific compounds listed above, and inhibiting the protein by at least about 10%, 20%, 30%, 40%, 50%, 60%, 70%, 80%, 90%, or 95%, or from about 50% to about 99%, or alternatively from about 20% to about 60%, and wherein said taste modulating protein is a naturally occurring taste modulating protein. In another embodiment, the present invention is directed to a method of inhibiting a taste modulating protein, comprising contacting said protein with a compound of Formula I, or any of the specific subclasses and specific compounds listed above, and inhibiting the protein by at least about 10%, 20%, 30%, 40%, 50%, 60%, 70%, 80%, 90%, or 95%, or from about 50% to about 99%, or alternatively from about 20% to about 40%, and wherein said protein is a naturally occurring human taste modulating protein.

In another embodiment, the present invention is directed to a nutriceutical composition comprising one or more nutriceuticals, one or more compounds according to Formula I, or any of the specific subgroups, or specific compounds described above, and optionally one or more carriers. Examples of nutriceutical compositions having an undesirable taste include, but are not necessarily limited to, enteral nutrition products for treatment of nutritional deficit, trauma, surgery, Crohn's disease, renal disease, hypertension, obesity and the like, to promote athletic performance, muscle enhancement or general well being or inborn errors of metabolism such as phenylketonuria. In particular, such nutriceutical formulations may contain one or more amino acids which have a bitter or metallic taste or aftertaste. Such amino acids include, but are not limited to, an essential amino acids selected from the group consisting of L isomers of leucine, isoleucine, histidine, lysine, methionine, phenylalanine, threonine, tryptophan, tyrosine, and valine. Additionally, the invention is directed to a process of preparing an improved nutriceutical composition, wherein the improvement comprises adding one or more compounds according to Formula I, or any of the specific subgroups, or specific compounds described above, to a nutriceutical composition. In certain embodiments, the one or more compounds according to Formula I, or any of the specific subgroups, or specific compounds described above, are added to a nutriceutical composition in an amount of about 1% to about 50%, or about 5%, 10%, or 15%, by weight.

In another embodiment, the present invention is directed to a dental hygienic composition comprising one or more compounds according to Formula I, or any of the specific subgroups, or specific compounds described above. Dental hygienic compositions are known in the art and include but are not necessarily limited to toothpaste, mouthwash, plaque rinse, dental floss, dental pain relievers (such as Anbesol™), and the like. For example, the invention includes a dental bleaching composition which comprises one or more compounds of Formula I, or any of the specific subgroups, or specific compounds described above, in an amount sufficient to inhibit a bitter taste. Dental bleaching compositions are known in the art. See, e.g., U.S. Pat. No. 6,485,709, which is herein incorporated by reference in its entirety. A dental bleaching composition of the present invention intended for use with dental trays may utilize a sticky carrier formed from a fluid and a thickener. The sticky carrier accordingly may comprise finely divided silica, such as silica fume, dispersed in a liquid, such as a polyol. Examples of suitable polyols include propylene glycol, glycerin, polypropylene glycols, sorbitol, polyethylene glycols and the like. While the carrier preferably includes thickeners, the carrier may also be only a liquid such as water or any of the liquid polyols without any thickeners.

Additionally, the invention is directed to a process of preparing an improved dental hygienic composition, wherein the improvement comprises adding one or more compounds according to Formula I, or any of the specific subgroups, or specific compounds described above, to a dental bleaching composition. In certain embodiments, the one or more compounds according to Formula I are added to a dental hygienic composition in an amount of about 1% to about 20%, preferably about 1% to about 5%, or about 5%, 10%, or 15%, by weight.

In another embodiment, the present invention is directed to a cosmetic product comprising one or more compounds according to Formula I, or any of the specific subgroups, or specific compounds described above. For example, but not by way of limitation, the cosmetic product comprising a compound according to Formula I, or any of the specific subgroups, or specific compounds described above, may be a face cream, lipstick, lipgloss, and the like. Other suitable compositions of the invention include lipbalm, such as Chapstick® or Burt's Beeswax® Lip Balm, further comprising one or more compounds according to Formula I, or any of the specific subgroups, or specific compounds described above.

Additionally, the invention is directed to a process of preparing an improved cosmetic product, wherein the improvement comprises adding one or more compounds according to Formula I, or any of the specific subgroups, or specific compounds described above, to a cosmetic product. In certain embodiments, the one or more compounds according to Formula I, or any of the specific subgroups, or specific compounds described above, are added to a cosmetic product in an amount of about 1% to about 20%, preferably about 1% to about 5%, or about 1%, 2%, or 3%, by weight.

In another embodiment, the present invention is directed to a food product comprising one or more compounds according to Formula I, or any of the specific subgroups, or specific compounds described above. Preferably, the food product is one which exhibits an undesirable taste, such as a bitter taste, which can be inhibited by a compound according to Formula I, or any of the specific subgroups, or specific compounds described above. Furthermore, in a preferred embodiment, the food product comprises a compound of Formula I, or any of the specific subgroups, or specific compounds described above in an amount sufficient to inhibit an unpleasant taste.

Specific food products and food ingredients to which one of more compounds of Formula I, or any of the specific subgroups, subclasses, or specific compounds described above, can be added include but are not necessarily limited to, potassium chloride, ammonium chloride, sodium chloride (e.g., table salt), magnesium chloride, halide salts, naringin, caffeine, urea, magnesium sulfate, saccharin, acetosulfames, aspirin, potassium benzoate, potassium bicarbonate, potassium carbonate, potassium nitrate, potassium nitrite, potassium sulfate, potassium sulfite, potassium glutamate, food preservatives in their physiologically acceptable salts, antibiotics, unsweetened chocolate, cocoa beans, yoghurt, preservatives, flavor enhancers, dietary supplements, gelling agents, pH control agents, nutrients, processing aids, bodying agents, dispersing agents, stabilizers, colorings, coloring diluents, anticaking agents, antimicrobial agents, formulation aids, leavening agents, surface active agents, anticaking agents, nutrient supplements, alkali, acids, sequestrants, denuding agents, general purpose buffers, thickeners, cooked out juice retention agents, color fixatives in meat and meat products, color fixatives in poultry and poultry products, dough conditioners, maturing agents, yeast foods, mold retardants, emulsifiers, texturizers, binders, water correctives, miscellaneous and general purpose food additives, tableting aids, lye peeling agents, washing water agents, oxidizers, antioxidants, enzymes, extenders, fungicides, cake mixes, coffee, tea, dry mixes, non-dairy creamers, salts, animal glue adjuvant, cheese, nuts, meat and meat products, poultry and poultry product, pork and pork products, fish and fish products, vegetable and vegetable products, fruit and fruit products, smoked products such as meat, cheese fish, poultry, and vegetables, whipping agents, masticatory substances in chewing gums, dough strengtheners, animal feed, poultry feed, fish feed, pork feed, defoaming agents, juices, liquors, substances or drinks containing alcohol, beverages including but not limited to alcoholic beverages and non-alcoholic carbonated and/or non-carbonated soft drinks, whipped toppings, bulking agents used in eatables including but not limited to starches, corn solids, polysaccharides and other polymeric carbohydrates, icings, as well as potassium-containing or metal-containing substances with undesirable tastes and the like.

Moreover, the present invention contemplates the preparation of eatables such as breads, biscuits, pancakes, cakes, pretzels, snack foods, baked goods etc. prepared using for example potassium bicarbonate or potassium carbonate in place of the sodium salts as leavening agents in conjunction with a compound according to Formula I, or any of the specific subgroups, or specific compounds described above, in an amount sufficient to eliminate one or more undesirable tastes. The compound according to Formula I, or any of the specific subgroups, or specific compounds described above, can be typically present in an amount ranging from about 0.001% to about 50% by weight, preferably about 0.1% to about 10% by weight, or alternatively, from 0.1% to about 1% by weight, of the material with the undesirable taste. The present invention also contemplates the preparation of preservatives for eatables comprising the potassium salts of benzoate, nitrate, nitrite, sulfate, and sulfite and so on, in conjunction with an appropriate concentration of a compound according to Formula I, or any of the specific subgroups, or specific compounds described above, to eliminate undesirable tastes in foodstuffs. Thus, the invention is directed to a process of preparing an improved food product, wherein the improvement comprises adding one or more compounds according to Formula I, or any of the specific subgroups, or specific compounds described above, to a food product. In certain embodiments, the one or more compounds according to Formula I, or any of the specific subgroups, or specific compounds described above, are added to a food product in an amount of about 1% to about 20%, preferably about 1% to about 5%, about 1%, 3%, or 4%, by weight.

In another embodiment, the present invention is directed to an animal food product comprising one or more compounds according to Formula I, or any of the specific subgroups, or specific compounds described above. The one or more compounds are preferably in an amount sufficient to inhibit one or more undesirable tastes associated with the animal food product. Animal food products are well known in the art, see, e.g., U.S. Pat. No. 6,403,142, and include dog food, cat food, rabbit food, and the like. The animal food product may also be food products useful for feeding livestock, such as cattle, bison, pigs, chicken, and the like. In another embodiment, the animal food composition of the present invention is a solid hypoallergenic pet food comprising a component that contains protein or protein fragments wherein all of said component is partially hydrolyzed and further comprises one or more compounds according to Formula I, or any of the specific subgroups, or specific compounds described above.

Additionally, the invention is directed to a process of preparing an improved animal food product, wherein the improvement comprises adding one or more compounds according to Formula I, or any of the specific subgroups, or specific compounds described above, to an animal food product. In certain embodiments, the one or more compounds according to Formula I, or any of the specific subgroups, or specific compounds described above, are added to an animal food product in an amount of about 1% to about 25%, about 1% to about 10%, or about 5%, 10%, or 15%, by weight.

In further embodiments of the present invention, any of the compositions described herein and containing a compound according to Formula I may further comprise one or more additional taste masking agents. Such masking agents include but are not limited to the group consisting of sucralose; zinc gluconate; ethyl maltol; glycine; acesulfame-k; aspartame; saccharin; fructose; xylitol; malitol; isomalt; salt; spray dried licorice root; glycyrrhizin; dextrose; sodium gluconate; sucrose; glucono-delta-lactone; ethyl vanillin; and vanillin.

In another embodiment, the present invention is directed to a composition comprising a compound of Formula I, or any of the specific subgroups, or specific compounds described above, and a carrier, wherein said carrier is suitable for an assay. Such carriers may include solid carriers and/or liquid carriers. A composition suitable for an assay may, but not necessarily, be sterile. Examples of suitable carriers for assays include dimethylsulfoxide, ethanol, dichloromethane, methanol, and the like. In another embodiment, a composition comprises a compound of Formula I, or any of the specific subgroups, or specific compounds described above, and a carrier, wherein the compound is in an amount suitable for inhibiting a taste modulating protein.

In each of the embodiments of the compositions described herein, a compound of Formula I, or any of the specific subgroups, or specific compounds described above, may be used in varying ratios to the agent that is believed to cause the unwanted taste, such as a bitter or sweet taste. For example, a composition of the invention may comprise a compound of Formula I in a molar ratio of about 1000:1 to about 1:1000, or alternatively administered in a molar ratio of about 500:1, about 200:1, about 10:1, about 1:1, about 1:10, about 1:200, or about 1:500, relative to the agent that is believed to cause the unwanted taste, such as a bitter or sweet taste. In another example, the present invention is directed to a food product comprising one or more food ingredients and a compound according to Formula I, wherein the molar ratio of the compound of Formula I to the food agent that causes, or is believed to cause, a bitter taste about 1000:1 to about 1:1000, or alternatively administered in a molar ratio of about 500:1, about 200:1, about 10:1, about 1:1, about 1:10, about 1:200, or about 1:500. As will be appreciated, the various ranges and amounts of the compound of Formula I can be used, with modifications if preferred, in each of the embodiments described herein.

The activity of a compound according to Formula I, or any of the specific subgroups, or specific compounds described above can be determined by testing said compound using a number of methods known in the art. For example, one can evaluate the ability of a compound to inhibit a bitter taste by using an in vivo taste assay. This in vivo assay identifies the bitter blockers that by testing their activity using human subjects. A concentration of the bitter compound quinine in water is found that the subject rates as 5 for bitterness on a scale of 0 to 10, where 0 is no bitterness and 10 is the most intense bitterness the subject has ever encountered. This concentration of quinine is then made up containing a concentration of a compound according to Formula I to be tested, and the subject rates the bitterness of this solution on the same scale.

The activity of a compound according to Formula I, or any of the specific subgroups, or specific compounds described above, can also be determined by means of the assay described in Example 6. The assay is described in complete detail in provisional application Ser. No. 60/732,636 (Attorney Docket No. 2305.0170000), which is incorporated by reference herein in its entirety.

The following examples are illustrative, but not limiting, of the method, compounds, and compositions of the present invention. Each of the compounds listed below was obtained from commercially available catalog companies, such as Aldrich RarechemLib, Aldrich Sigma, AlsInEx, Biotech Corp., Brandon/Berlex, Calbiochem, ChemBridge, Comgenex West, Foks H, G. & J. Research, IBS, ICN Biochemicals, Institute for Chemotherapy, Kodak, Lederle Labs, Ligand-CGX, Maybridge PRI, Menai Organics, Menai/Neurocrine, MicroSource, MPA Chemists, Mybrgd/ONYX, PRI-Peakdale, RADIAN, Receptor Research, RGI, Rhone-Poulenc, SPECS/BioSPECS/SYNTHESIA, T. Glinka, Tripos Modern, VWR, Zaleska, Zelinksy/Berlex, Aeros, and Chemica. The compounds were purified using conventional purification procedures, such as HPLC. The identity of the compound was confirmed using HPLC and mass spectrometry. It is understood that the invention includes all stereoisomers. Other suitable modifications and adaptations of the variety of conditions and parameters normally encountered and obvious to those skilled in the art are within the spirit and scope of the invention.

### EXAMPLES

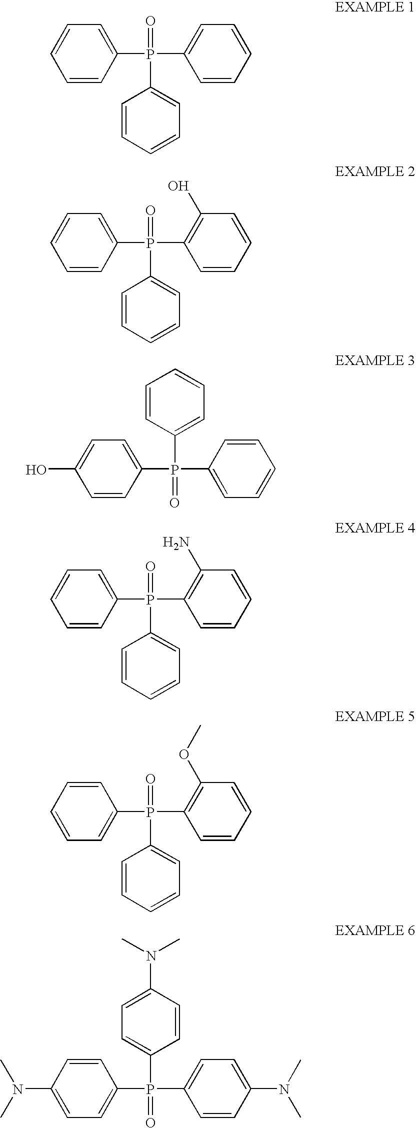

### Activity of Selected Compounds

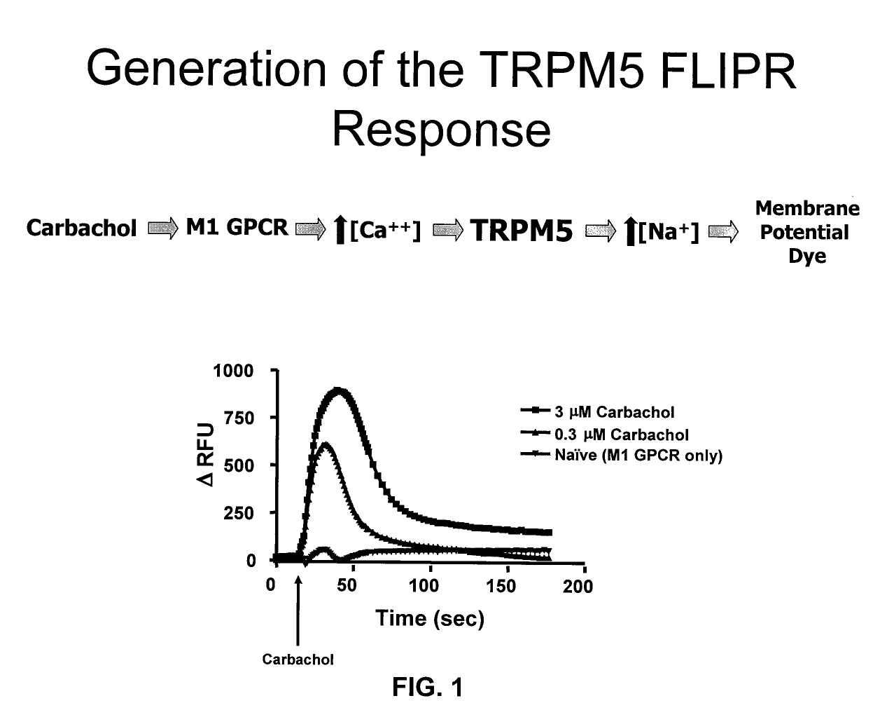

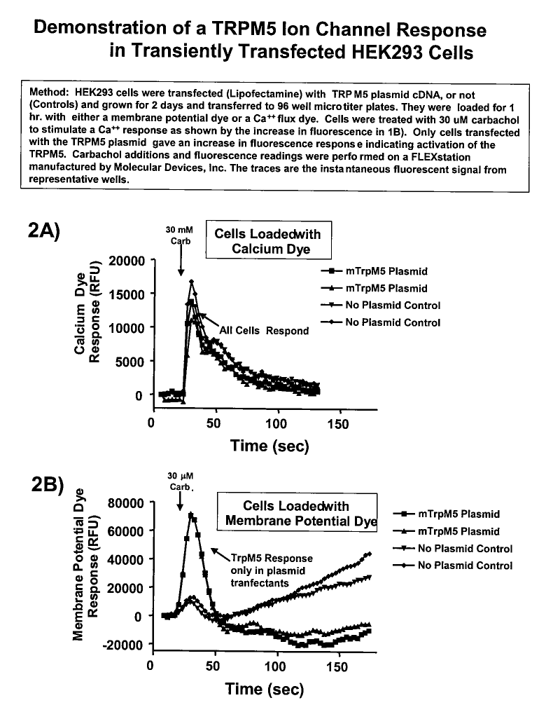

The activity of human TRPM5 ion channel was measured in live cells on a fluorescent imaging plate reader (FLIPR). The basis of the assay (shown in FIG. 1) is the calcium-dependent activation of the ion channel which occurs by activation of a G-protein coupled receptor (GPCR). GPCR activation by an appropriate agonist causes a transient increase in intercellular Ca²⁺ ion concentration which in turn causes the ion channel to open, letting in Na⁺ ions. This influx causes a change in the membrane potential of the cell which can be monitored as a change in the fluorescent signal from voltage-dependent (membrane potential) fluorescent dyes. A demonstration of the assay is shown in FIGS. 2A and 2B, where traces of fluorescent response (Ex 530 nm/Em565 nm) versus time are shown for cells containing the plasmid and sham plasmid controls. While all cells gave a Ca²⁺ response to the endogenous muscarinic GPCR agonist carbachol (upper panel), only cells containing the plasmid showed a sharp peak for the membrane potential dye response (lower panel).

For the screening assay, the human TRPM5 gene was cloned, put into HEK293 cells, and a stable, high expression clone was used for screening. Cells were grown in standard media at 37° C. The day before screening, the cells were removed from flasks and added to 384 well clear bottom plates (8K cells in 20 μL/well). On the assay day, 20 μL of membrane potential dye (Part No. R8123, Molecular Devices Corp.) was added to the cells and dye was allowed to be taken up, i.e., load, into the cells for 1 hr at 37° C. The dye-loaded cell plate was placed in the FLIPR along with a second 384 well plate containing test compounds as well as positive (fully inhibited) and negative (non-inhibited) controls. The assay was started by addition of 10 μL of solution from the compound plate into the cell plate. During this process, continuous fluorescent recordings were made simultaneously for all wells. After addition of the compound solution, the tips were automatically washed and a stimulation solution of 3 μM ATP (an agonist for an endogenous purinurgic GPCR, was added to all wells of the cell plate. The height of the response was calculated and percent inhibition values, versus negative control wells, was calculated for the test samples.

Unless otherwise indicated, the data in the table below were determined using the three assays described above, providing percent inhibition data at 10 μM.

| | IC50 |
|---|---|
| Example No. | (μM) |
| 1 | 5 |
| 2 | 5 |
| 3 | 5 |
| 4 | 5 |
| 5 | >100 (Not active) |
| 6 | >100 (Not active) |

Having now fully described this invention, it will be understood by those of ordinary skill in the art that the same can be performed within a wide and equivalent range of conditions, formulations and other parameters without affecting the scope of the invention or any embodiment thereof. All patents and publications cited herein are fully incorporated by reference herein in their entirety.

What is claimed is:

1. A method of inhibiting a taste modulating protein, comprising contacting said protein with a compound of Formula I: 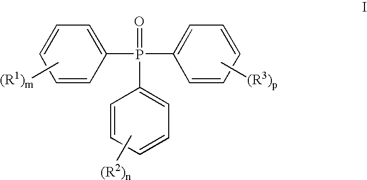
or a physiologically acceptable salt thereof, wherein
R¹, R² and R³ are independently selected from the group consisting of C₁₋₆ alkyl, C₂₋₆ alkenyl, C₂₋₆ alkynyl, hydroxy, C₁₋₆ alkoxy, amino, C₁₋₆ alkylamino, C₁₋₆ dialkylamino, and halo; and
m, n, and p are independently an integer from 0 to 5.

2. The method according to claim 1, wherein R¹, R², and R³ are hydrogen.

3. The method according to claim 1, wherein R¹ and R² are hydrogen.

4. The method according to claim 1, wherein at least one of R¹, R², and R³ is hydroxy.

5. The method according to claim 1, wherein R³ is an amino group.

6. The method according to claim 1, wherein R³ is methoxy.

7. The method according to claim 1, wherein R³ is diethylamino.

8. The method according to claim 1, wherein the compound of Formula I is

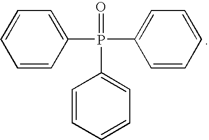

9. The method according to claim 1, wherein the compound of Formula I is

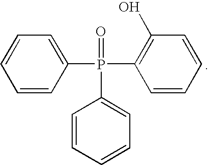

10. The method according to claim 1, wherein the compound of Formula I is

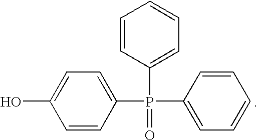

11. The method according to claim 1, wherein the compound of Formula I is

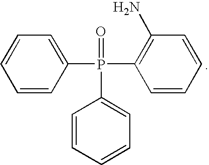

12. The method according to claim 1, wherein the compound of Formula I is

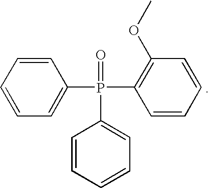

13. The method according to claim 1, wherein the compound of Formula I is

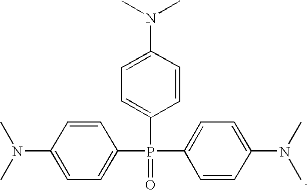

14. The method according to claim 1, wherein the protein is a nonhuman TRPM5 protein.

15. The method according to claim 1, wherein the protein is a cow, horse, sheep, pig, cat, dog, rabbit, or monkey TRPM5 protein.

16. The method according to claim 1, wherein the protein is a human TRPM5 protein.

17. The method according to claim 1, wherein the compound according to Formula I is administered as a pharmaceutical composition.

18. The method according to claim 1, wherein the compound according to Formula I is administered as a veterinary composition.

19. The method according to claim 1, wherein the compound according to Formula I is administered as a food composition.

20. The method according to claim 1, wherein the compound according to Formula I is administered as a dental hygienic composition.

21. The method according to claim 1, wherein the compound according to Formula I is administered in a concentration from about 1% to about 10% on a weight percentage basis.

22. The method according to claim 1, wherein the compound according to Formula I is administered in an amount of about 0.01 mg to about 10 mg.

23. The method according to claim 1, wherein said receptor is in vitro.

24. A method of inhibiting a taste, comprising administering to a subject in need of said taste inhibiting one or more compounds of Formula I: 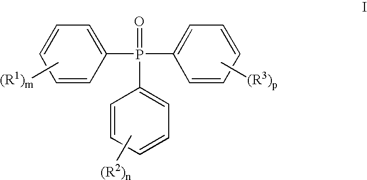
or a physiologically acceptable salt thereof, wherein
R¹, R² and R³ are independently selected from the group consisting of C₁₋₆ alkyl, C₂₋₆ alkenyl, C₂₋₆ alkynyl, hydroxy, C₁₋₆ alkoxy, amino, C₁₋₆ alkylamino, C₁₋₆ dialkylamino, and halo; and
m, n, and p are independently an integer from 0 to 5;
wherein said compound is administered in an amount sufficient to inhibit said taste.

25. A method of inhibiting the depolarization of a taste receptor cell, comprising contacting said taste receptor cell with one or more compounds of Formula I: 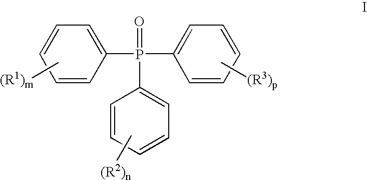
or a physiologically acceptable salt thereof, wherein
R¹, R² and R³ are independently selected from the group consisting of C₁₋₆ alkyl, C₂₋₆ alkenyl, C₂₋₆ alkynyl, hydroxy, C₁₋₆ alkoxy, amino, C₁₋₆ alkylamino, C₁₋₆ dialkylamino, and halo; and
m, n, and p are independently an integer from 0 to 5.

26. A pharmaceutical composition comprising one or more pharmaceutically acceptable carriers and one or more compounds according to Formula I: 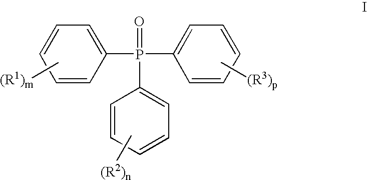
or physiologically acceptable salt thereof wherein
R¹, R² and R³ are independently selected from the group consisting of C₁₋₆ alkyl, C₂₋₆ alkenyl, C₂₋₆ alkynyl, hydroxy, C₁₋₆ alkoxy, amino, C₁₋₆ alkylamino, C₁₋₆ dialkylamino, and halo; and
m, n, and p are independently an integer from 0 to 5.

27. A method of preparing an improved pharmaceutical composition, wherein the improvement comprises adding to a pharmaceutical composition one or more compounds according to Formula I: 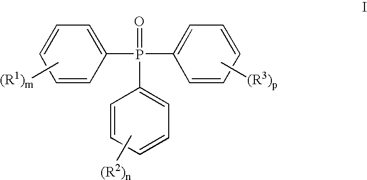
or physiologically acceptable salt thereof wherein R¹, R² and R³ are independently selected from the group consisting of C₁₋₆ alkyl, C₂₋₆ alkenyl, C₂₋₆ alkynyl, hydroxy, C₁₋₆ alkoxy, amino, C₁₋₆ alkylamino, C₁₋₆ dialkylamino, and halo; and
m, n, and p are independently an integer from 0 to 5.
